(19) United States
(12) Patent Application Publication
(10) Pub. No.: US 2015/0198597 A1
(43) Pub. Date: Jul. 16, 2015

(54) Method for monitoring PARP activity in cells by PARP activation

(71) Applicant: Stichting Het Nederlands Kanker Instituut, AMSTERDAM (NL)

(72) Inventors: Conchita Vens, Amsterdam (NL); Derk Pluim, Amsterdam (NL); Marcel Verheij, Amsterdam (NL); Johannes Henricus Matthias Schellens, Amsterdam (NL)

(21) Appl. No.: 14/420,065

(22) PCT Filed: Aug. 8, 2013
(86) PCT No.: PCT/NL2013/050587

§ 371 Date: Feb. 6, 2015

Related U.S. Application Data
(60) Provisional application No. 61/680,904, filed on Aug. 8, 2012.

(30) Foreign Application Priority Data
Aug. 8, 2012 (NL) 2009296

(51) Int. Cl. G01N 33/573

(52) U.S. Cl. CPC G01N 33/573; G01N 2333/91142

(57) ABSTRACT

The current invention provides for improved methods for determining PAR, and uses thereof. These improved methods involve an incubation step at a low temperature, for example around, but above, the freezing point after a PARP activation step. This improves the assay's sensitivity in comparison to current assay's performance. For example, sensitivity can be improved in methods assaying cells obtained from subjects such as human patients. The current invention also allows to provide methods to screen compounds for modulating PARG and/or PARP activity.

The poly-(ADP)-ribose polymerases (PARP) enzymes belong to a family of proteins that are mainly involved in DNA repair and programmed cell death. The family of poly-ADP-ribosylating PARP enzymes that have been identified in mammalians so far includes PARP1, PARP2, PARP3, PARP4/vPARP, PARP5/Tankyrases-1 and PARP6/Tankyrases-2 (Hassa and Hottiger, Front Biosci. 2008 Jan. 1; 13:3046-82; Messner and Hottiger, Trends Cell Biol. 2011 September; 21(9):534-42; and Hottiger et al. Trends Biochem Sci. 2010 April; 35(4):208-19). PARP1 and 2 are normally found in the cell's nucleus, wherein its main function is to detect and signal DNA damage, in particular DNA nicks and DNA single strand breaks. Upon recognition, PARP aids the enzymatic machinery involved in DNA single strand break repair by synthesizing poly-(ADP)-ribose chains (PAR). Auto-poly(ADP)ribosylation of PARP itself and PAR formation on chromatin in the vicinity of the DNA lesions act as a signal for other DNA repair enzymes. PAR formation is counteracted by the poly-(ADP)-ribose glycohydrolase (PARG) activity that results in the degradation of the PAR chains.

Since NAD+ is required for generating the ADP-ribose monomers, the induction of PARP activity results in a decrease of NAD+ levels in the cell. Hence, NAD+ consumption by the induction of PARP activity can affect cellular metabolism and limit glucose oxidation, which also requires NAD+, and thus may reduce the availability of ATP, the energy source of the cell. NAD+ depletion as a consequence of extensive PARP activity has been associated with cell death.

The induction of DNA damage is instrumental in cancer therapies, namely chemo- and radiotherapy. Other conditions such as inflammation, (reperfusion) ischemia and neuro-degeneration have been associated with the occurrence of DNA lesions. Normal cellular metabolic activity and oxidative stress also cause DNA breaks to some extent that require repair to assure cellular survival and proliferation. As PARP enzymes have a pivotal role in the signalling and repair of such lesions, they have been identified as a promising target for drug development. This drove the development of specific PARP inhibitors that block the poly(ADP)ribosylation activity. Several novel compounds with specific PARP inhibitory activity exceeded preclinical testing and are currently evaluated in a clinical setting (Verheij, Vens, van Triest, Drug Resist Updat 13, 29-43, 2010).

PARP inhibitors have shown single agent activity in the treatment of a subset of breast or ovarian cancers. Tumours with defects in DNA damage repair, the homologous recombination double strand break repair, are particularly sensitive to these PARP inhibitors warranting their use in such a setting. BRCA2 mutation carriers for example often develop cancers with such defects. Due to its involvement in DNA repair, inhibition of PARP causes hypersensitivity to chemotherapeutic agents or radiation. Hence, PARP inhibition is believed to improve chemo- and radiation therapies. Current clinical trials evaluate such combination strategies. Administration of specific PARP inhibitors is also considered in other settings such as in the treatment of inflammatory diseases.

Crucial for the assessment of the benefit of a treatment with any PARP inhibitor in any setting is the sensitive detection of dose dependent and/or inter-individual variations in the inhibitory activity of these novel agents. In order to evaluate the efficacy of PARP inhibitors, assays have been developed that can monitor the activity of the PARP enzyme. For example, Trevigen® (Gaithersburg, Md., USA) has developed enzyme-linked immunosorbent assays (ELISA) with which the product of PARP activity, PAR, can be measured. Hence, the activity of PARP is monitored by measuring the level of PAR in a sample. Commercial assays that are available are from Trevigen® and include for example the assays 4676-096K, 4677-096K, 4684-096K, 4685-096K, 4690-096K, and 4520-096K, which all can be used to monitor PARP activity (see www.trevigen.com). These types of assays have limitations. The limit for accurate and robust PAR detection in these assays is often above the level that is typically present in untreated cells from subjects, e.g. human subjects (Liu et al., 2008, Anal Biochem; October 15; 381(2):240-7). Hence, it is difficult to accurately quantify a reduction in PAR levels. This is in particular relevant when cells and/or subjects have been treated with PARP inhibitors which requires a sensitive assessment of PAR levels below the detection limit for most subjects. This limitation restricts the applicability of such assays.

### SUMMARY OF THE INVENTION

The current invention provides for improved methods for determining PAR, and uses thereof. The invention relates to additional steps that can be included in assays that are used to determine and quantify intracellular PAR. These additional steps can highly improve the assay's sensitivity in comparison to current assay's performance. This is in particular the case when assaying cells that have been obtained from subjects such as human patients. One of the steps is a step that activates PARP, i.e. a step that induces DNA damage. DNA damage induction can include DNA single-strand and DNA double-strand breaks, e.g. by exposure of cells to ionizing radiation such as from a gamma ray source. This step can be included in the procedure for sample preparation prior to PAR detection and analysis such as with an ELISA based assay. This additional step activates PARP enzymes within the cells, thereby elevating the PAR levels to amounts that allow sensitive quantification, in particular in cells obtained from blood samples from a subject. The other step involves an incubation step, wherein cells are incubated at a low temperature, for example around but above the freezing point but below 37° Celsius (° Celsius may also be referred to as ° C.), preferably below 25° Celsius, preferably below 10° Celsius, most preferably below 4°. The incubation step may be a separate step from the PARP activation step but may also be combined with the PARP activation step. The PARP activation step may also be carried out at a temperature below 37° Celsius, preferably below 25° Celsius, preferably below 20° Celsius, preferably below or at most 10° Celsius, most preferably below 4° Celsius. By having the cells at a temperature below 37° Celsius, preferably below 25° Celsius, preferably below 20° Celsius, preferably below or at most at 10° Celsius, most preferably below 4°, an even higher and steady level of PAR can be achieved, therefore aiding the reliable and sensitive PAR detection and improving the detection sensitivity and/or minimizes variability that may be caused by rapid alterations in PAR levels (PAR degradation) at higher temperatures. These additional steps can be incorporated in any assay for detection of PAR. Such assays for detection of PAR are suitable for monitoring PARP (or PARG) activity intra-cellularly, e.g. in the presence or absence of a PARP inhibitor. The current invention allows for an increase of PAR levels in cells to levels above the detection limit, to allow a quantitative analysis and comparison of PAR between different subjects and/or different conditions. The current invention allows assessing effects and/or toxicity of a PARP inhibitor and/or radiation in the treatment of a subject. Such an analysis may assist in the selection of a suitable dose of PARP inhibitors and/or radiation in subjects.

### FIGURES

FIGS. 1-8 are graphs depicting the results that were obtained following the experimental steps such as outlined in the example section.

FIG. 1: PAR levels and PARP activity induction by ex vivo radiation.

Peripheral blood mononuclear cells (PBMC) derived from human blood samples were exposed to 8 Gy of gamma rays or not exposed. Cells were irradiated on ice (approximately 0-4° Celsius) and cells were subsequently incubated at 37° Celsius for different time periods (x-axis). Cells were lysed and samples prepared according to the manufactures protocol. PAR levels were measured (y-axis) in an ELISA based PAR assay (HT PARP in vivo Pharmacodynamic Assay II, Trevigen®). A strongly elevated level of PAR was detected in the cells exposed to 8 Gy at early timepoints, which reduced with time. After 20 minutes (min) PAR levels in the radiated cells were 2-3 fold higher than the PAR levels in unexposed cells.

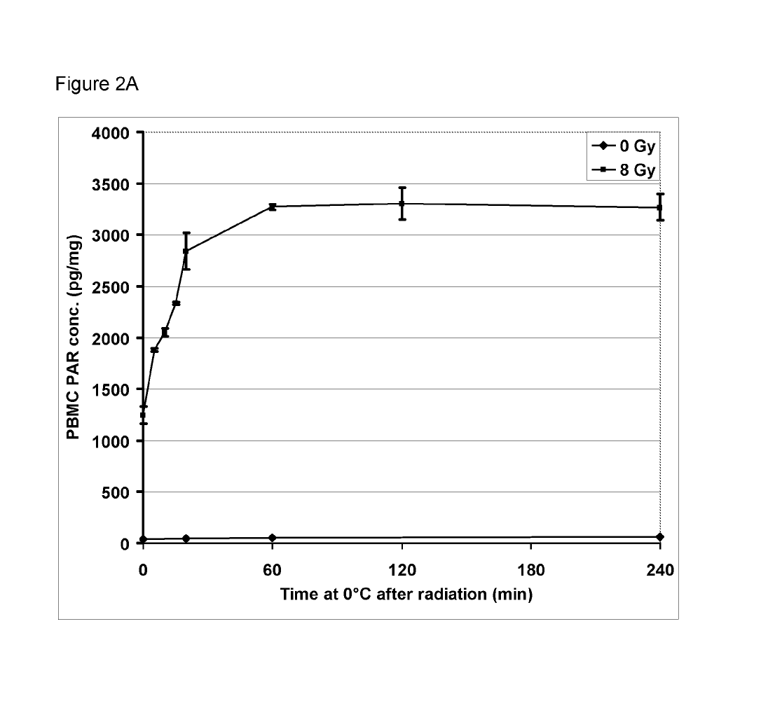

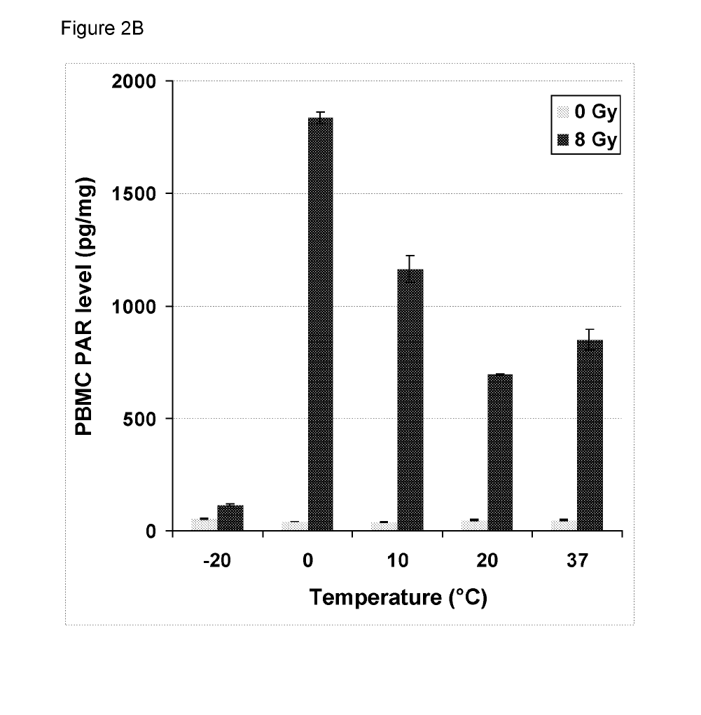

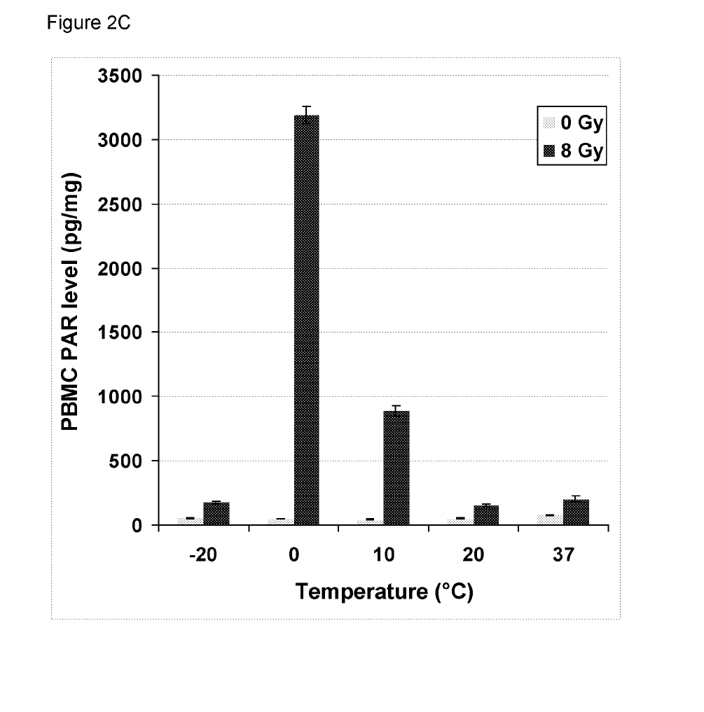

FIG. 2. Stabilization of PAR levels upon PARP activation.

A) PBMCs were exposed to 8 Gy of gamma rays or not exposed. Cells were irradiated while incubated on ice (at 0-4° Celsius) and cells were subsequently incubated on ice (at 0-4° Celsius) for the given different time periods (0, 5, 10, 15, 20, 60, 120, 240 minutes (x-axis)). Cells were immediately lysed thereafter and PAR levels were measured by using the Trevigen® PARP Pharmacodynamic assay II (y-axis). A strongly elevated level of PAR was detected in the cells exposed to 8 Gy. Samples have been diluted accordingly to allow measurement within the detection range of the assay. PAR levels increased with time and maximum values were reached after about 60 minutes. Values were reached that are about 100-fold higher than in the unexposed cells.

B and C) PBMCs were exposed to 8 Gy of radiation or not exposed. Cells were irradiated while incubated on ice and subsequently maintained at different temperatures (−20, 0, 10, 20 and 37 degrees Celsius) for 10 min. (B) or 60 min. (C). Cells were immediately lysed thereafter and PAR levels (y-axis) were measured by using the Trevigen® PARP Pharmacodynamic assay II. PAR levels are elevated in cells exposed to 8 Gy. Maintenance of these high levels over time are improved by low temperatures. At all temperatures values are higher than in the unexposed cells.

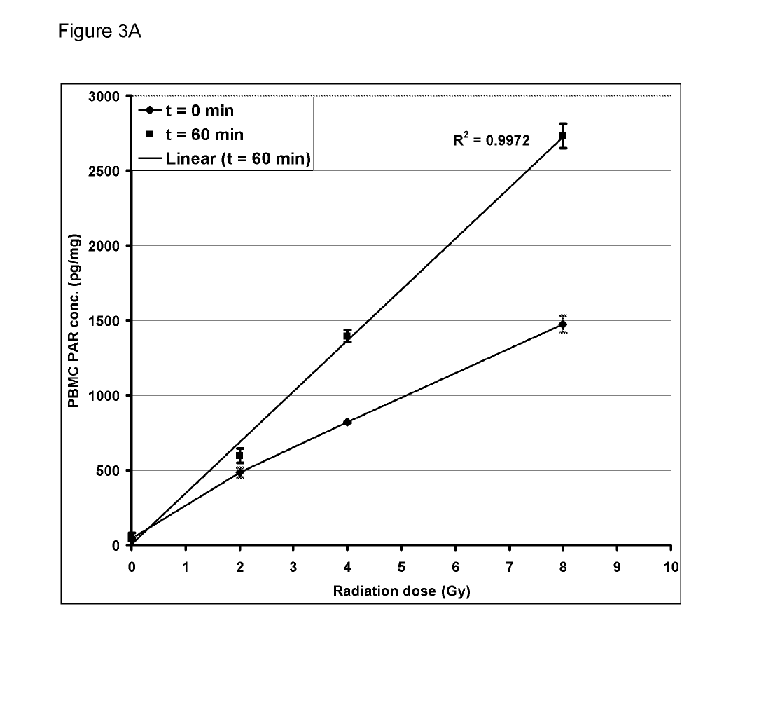

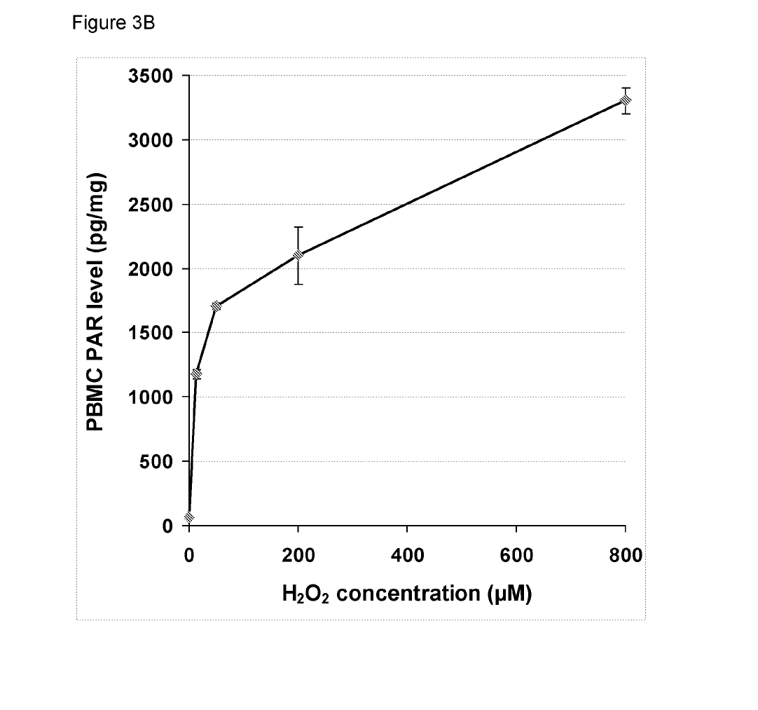

FIG. 3. Linearity of PARP activity/PAR level induction.

A) PBMCs were exposed to different doses of gamma rays, 0, 2, 4 and 8 Gy. Cells were irradiated on ice (at 0-4° Celsius) and cells were maintained on ice (at 0-4° Celsius) for either 0 minutes or 60 minutes. The PAR concentration (y-axis) is plotted against the radiation dose (x-axis). A linear relationship was found between radiation dose and PAR levels. The 1 hour incubation time on ice increased the slope while maintaining similar intra-sample variability, therefore resulting in an even further improved sensitivity.

B) PBMCs were exposed to different concentrations of hydrogen peroxide (H₂O₂) at 0, 12.5, 50, 200 and 800 μM. Cells were treated on ice (at 0-4° Celsius) and cells were maintained on ice (at 0-4° Celsius) for 60 minutes. Cells were immediately lysed thereafter and PAR levels were measured by using the Trevigen® PARP Pharmacodynamic assay II. The PAR levels (y-axis) are plotted against the H₂O₂ concentration (x-axis). Higher PAR levels were found with increasing H₂O₂ treatment concentration thereby demonstrating efficient PAR induction by PARP activating compound, i.e. chemicals and genotoxins that induce DNA lesions.

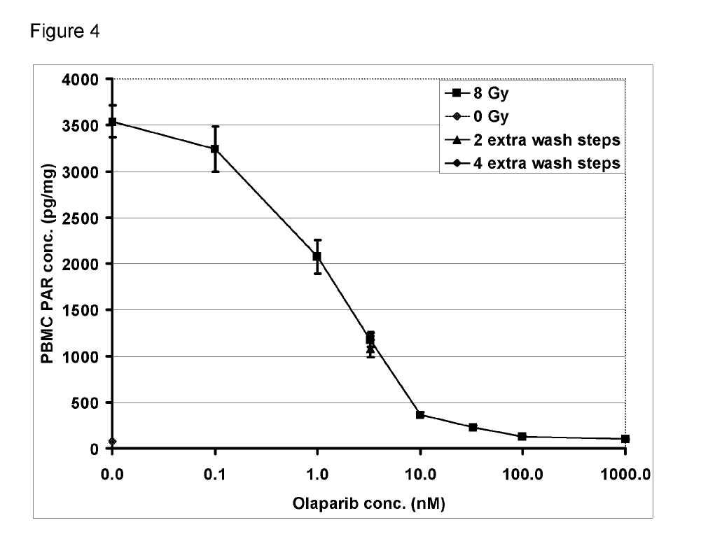

FIG. 4. Sensitive determination of PARP activity inhibition.

PBMCs were incubated in the presence of Olaparib, a PARP-1 inhibitor at different concentrations (x-axis). PBMCs were incubated with Olaparib in their original blood plasma in 0.1, 1, 3.3, 10, 33, 100, and 1000 nM Olaparib for 1 h at 37° Celsius. Then, PBMCs were washed with ice-cold phosphate buffered saline (PBS). This was followed by 8 Gy gamma radiation on ice and a post-incubation period on ice for 1 h. Results are given as mean±SD of three independent samples.

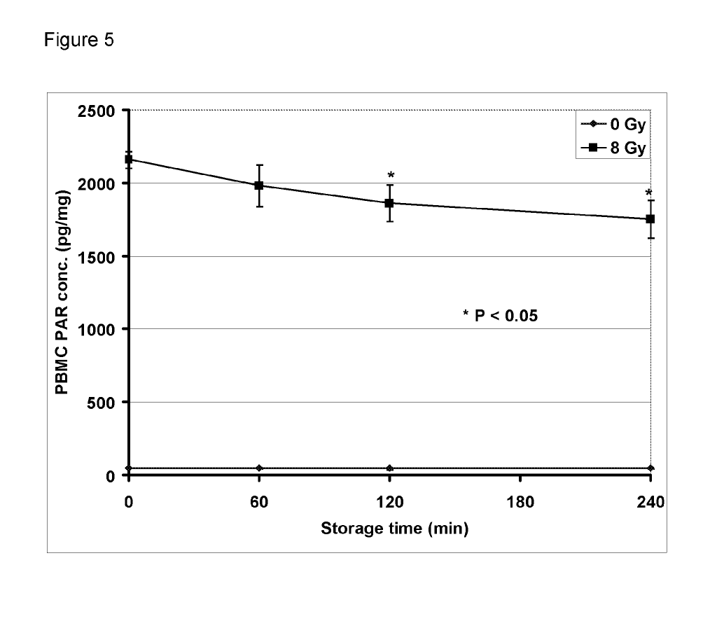

FIG. 5. Stability of PARP in PBMC isolated from whole blood stored at room temperature (RT).

Whole peripheral blood from a healthy volunteer was stored for the indicated time (x-axis) at RT before isolation of the PBMCs. After this incubation (storage) period PBMCs were irradiated with a dose of 8 Gy on ice and subsequently incubated for 1 h on ice (0-4° Celsius) before sample preparation for PAR level determination (y-axis). PARP activity was stable for at least 60 minutes, but a significant (P=0.021) drop of 14% in PAR levels was found after a storage time of the blood of 120 minutes, which increased to 19% (P=0.008) after 240 minutes. Results are given as mean±SD of three independent samples.

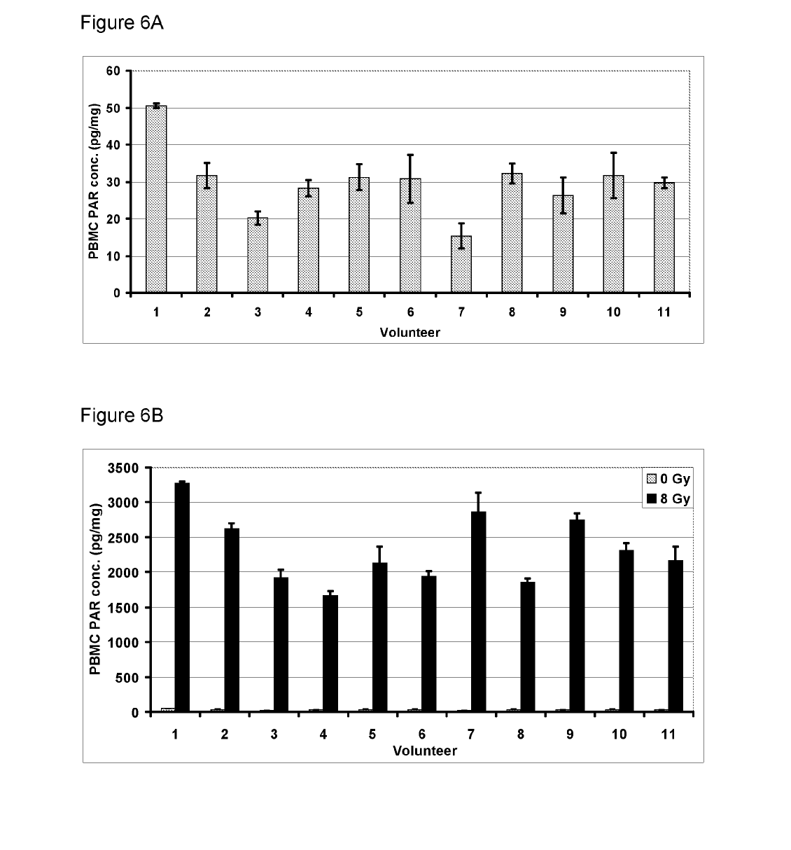

FIGS. 6A and 6B. Inter- and intra-individual PAR and PARP activity variability. PAR levels from 11 different human subjects with ex vivo radiation (8 Gy) and without (0 Gy) were compared. As previously reported, the basal PAR levels without the radiation step range from 15-50 pg/mg (y-axis, FIG. 6A). When cells of the subjects were exposed to radiation, levels of PAR ranged from about 1500-3300 pg/ml (y-axis, FIG. 6B). The PBMCs were irradiated and incubated for 1 hour on ice prior sample preparation according to the Trevigen® sample preparation protocol.

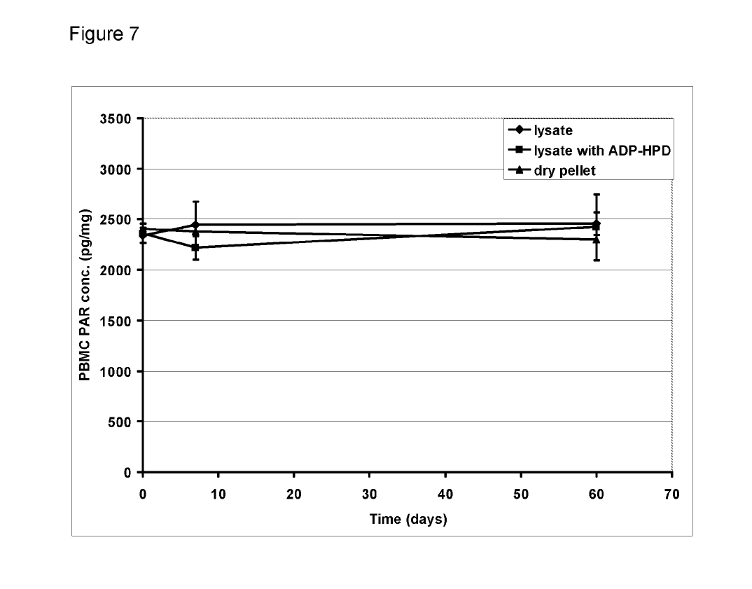

FIG. 7. Stability of PAR levels at three different conditions.

PBMCs were irradiated at 8 Gy on ice and subsequently incubated for 1 h on ice. PBMCs were stored after centrifugation and removal of the supernatant as a cell pellet or as a lysate with or without the addition of ADP-HPD to 1 μM (PARG inhibitor). The cell pellet samples were lysed just before the analysis of PAR levels (y-axis) on the indicated days (x-axis). No significant degradation of PAR was found using these three storage methods during the tested 60 days of storage. Results are given as mean±SD of three independent samples.

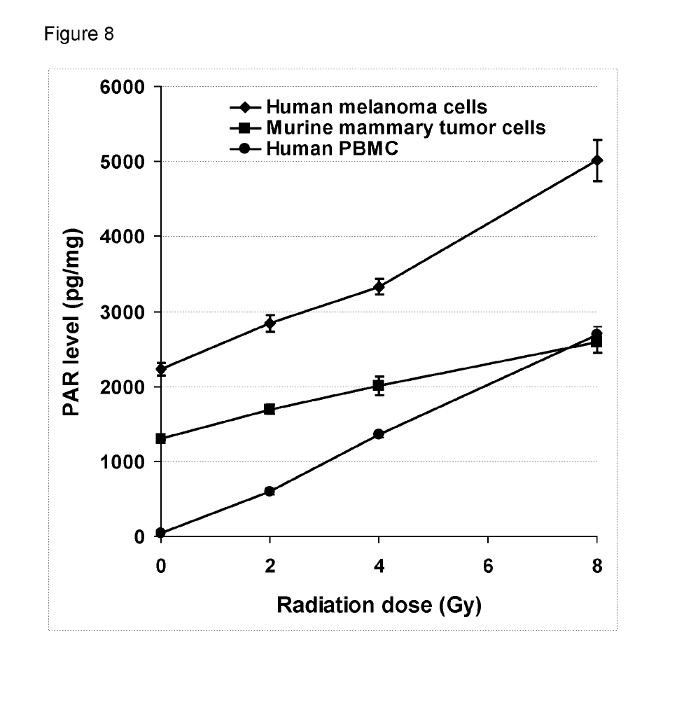

FIG. 8. PAR level induction in cell lines

Cultured murine mammary tumor cells, cultured human melanoma tumor cells and human PBMCs were exposed to 0, 2, 4 or 8 Gy while on ice. Cells were subsequently maintained on ice for 60 min. Cells were immediately lysed thereafter and PAR levels (y-axis) were measured by using the Trevigen® PARP Pharmacodynamic assay II. PAR levels in the unexposed cultured tumor cells are higher than in the unexposed human PBMCs. In all cells higher PAR levels were found with increasing irradiation dose.

### DESCRIPTION OF THE INVENTION

The PARP enzyme is an important target in various diseases and treatments thereof. It is thus of importance to be able to determine the activity of the PARP enzyme well in cells obtained from subjects. These subjects are for example treated or are candidates to be treated with PARP inhibitors. It is understood that with determining PAR in cells is not meant determining the presence or absence of PAR, but that it relates to determining the PAR quantity in cells, i.e. PAR levels. By determining PAR in cells, the efficacy of a PARP inhibitor can be determined. PARP inhibitors have emerged as a promising therapeutic class of compounds, and may include Olaparib, Veliparib, PF-1367338, MK-4827 and CEP-9722 (Gartner et al., Practice of Oncology 2008, Vol16 Nr2 pp 83-90; and Penning, Curr Opin Drug Discov Devel. 2010 September; 13(5):577-86). Cellular PAR levels correlate with PARP activity; hence PARP activity can be determined by measuring the quantity of PAR. The measurement of PAR is an indirect measurement of the activity of PARP as the product of PARP activity is PAR, i.e. poly(ADP) ribosylated proteins, which can be detected, for example by antibodies. It has been found by the inventors and others that, when preparing samples and measuring PAR in cells according to current protocols that are available, the levels of PAR are often too low for a reliable quantification, in particular in cells obtained from a subject. Levels of PAR, and hence PARP activity, determined in such assays are in a considerable fraction of samples below the detection or quantification limit of the available assays (see FIG. 6A, detection limit of 20 pg/mg). Hence, this allows at most, if at all, only a qualitative analysis of PAR, and hence also of PARP activity. The current assays are thus not suitable to reliably determine PAR levels, in particular in cells obtained from a subject. Importantly, by the action of PARP inhibitors, the amounts of PAR would decrease even further resulting in a concentration of PAR far below the detection limit. This may prohibit an accurate quantification of the activity of compounds that alter PAR formation (PARP or PARG activity). For example, as shown in FIG. 6A, the maximum amount of PAR measured in cells from 11 different patients is about 50 pg/mg, with a detection limit of 20 pg/mg. Hence, in this example, already a three-fold inhibition by a PARP inhibitor would result in unreliable PAR quantification making it impossible to assess the activity of a PARP inhibitor quantitatively. Inter-individual variation in the ability to activate PARP or the inter-individual or compound dependent efficacy of PARP inhibition can thus not be assessed by the assays applying current procedures. The inventors now provide for an improved method for monitoring PARP activity in cells, which method comprises the step of activating PARP in the cells such that PAR levels are increased in the cell. PARP activation may be achieved by inducing DNA damage, such as DNA lesions, in the cells. Treatments that can induce such DNA damage are well known to the skilled person, such as incubating the cells with DNA damaging agents or by exposing cells to ionizing radiation. As a consequence, the amount of PAR in the cell is significantly increased, e.g. well above the detection limit, allowing a quantitative and reliable analysis of PARP activity in the cells, in particular in cells obtained from a subject.

It is understood that by determining PAR in any of the methods of the invention, the activity of PARP and PARG can be monitored. It is understood that monitoring the activity of PARP, comprises the activity of the family of PARP enzymes, which may include PARP1, PARP2, PARP3, PARP4/vPARP, PARP5/Tankyrases-1 and PARP6/Tankyrases-2. Preferably, monitoring the activity of PARP according to the invention relates to the monitoring of PARP-1 and PARP-2.

The current invention provides for an improved method for determining PAR in cells ex vivo comprising the steps of:

providing cells ex vivo;

activating PARP in the cells;

incubating the PARP activated cells;

determining the quantity of PAR in the incubated PARP activated cells;

wherein the cells are kept at a temperature below 37° Celsius during the incubation step and, optionally, during the PARP activation step.

The provided cells can be any cells for which it is of interest to monitor PARP activity. For example, cell lines may be of interest. Cell lines may be immortalized cells, e.g. derived from cancer cells. Cell lines may be derived from any species, e.g. animals or human, for which it is of interest to determine PAR, to monitor PARG and/or PARP activity. Preferably, the cells are obtained from a subject. With cells obtained from a subject it is understood that the cells are not cell lines, i.e. they are not immortalized cells, but that the cells are obtained from the subject and are kept ex vivo for a limited period of time. Such cells may be also be referred to as primary cells. Providing cells ex vivo is understood to include also material and tissue from biopsies and/or explants biopsies or other samples obtained from an organism, as long as the cells ex vivo are not comprised within the organism, i.e. in vivo. Hence, (primary) cells that were in vivo can be provided ex vivo to carry out the methods of the invention. Provided cells ex vivo may be prepared such that the cells dissociate, e.g. by using trypsin. The ex vivo cells may be cultured, for example for 1-2 weeks, wherein the cells may multiply, i.e. divide. It may be advantageous to have the cells not dividing, hence the time period wherein the cells are ex vivo may preferably be at most 2 days, at most 1 day, at most 8 hours (may also be referred to as h), at most 6 hours, at most 2 hours, or at most 1 hour. Keeping the cells for a short time ex vivo is for example advantageous when PARP activity and/or PARG activity is monitored in a patient. The patient can for example be treated with a PARP and/or PARG inhibitor, and by having the cells ex vivo for short time before carrying out the method of the invention, the condition of the cells may reflect the in vivo condition as close as possible. The cells obtained from a subject can be from any subject for which it is of interest or use to determine PAR, e.g. for monitoring PARP activity or PARG activity. The subject according to the invention can be a vertebrate, preferably a mammal. The mammal can be a human or an animal, e.g. a domesticated animal. Preferably the subject is a human or a human patient. The cells that are provided from the subject can be used directly, or alternatively can be stored until they will be used. When cells are stored, this may be done as soon as possible after the cells have been obtained from the subject, for example, preferably within at most 8 hours, at most 6 hours, at most 2 hours, or within preferably at most 1 hour. When the cells are stored the person skilled in the art understands that conditions may be selected that do not substantially affect PARP activity and that may allow the cells to substantially remain intact.

The cells that are provided are subjected to PARP activation. PARP activation is preferably an active step, i.e. wherein an outside agent or outside PARP activation inducer such as irradiation is applied. PARP activation can also be induced passively, e.g. by keeping the cells ex vivo for some time may also activate PARP, however, such a passive PARP activation may not be preferred as it is much less well controlled as compared to active PARP activation steps.

The step of PARP activation preferably is at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius. For example and in one embodiment, the step of PARP activation comprises irradiating the cells with a source of ionizing radiation. Ionizing radiation is radiation composed of particles or photons, i.e. electromagnetic waves that can liberate an electron from an atom or molecule, thereby producing ions. Ionizing radiation can be composed of particles or photons with energies above about 10 electron volts (eV). Electromagnetic waves from ultraviolet wave length and below can reach such energy levels, e.g. such as X-rays and gamma rays. Electromagnetic radiation, particle radiation with neutrons, protons, alpha particles or beta particles can also be ionizing radiation. Suitable sources of ionizing radiation are for example cesium-137, iridium-192 and cobalt-60. In a normal environment, there is always ionizing radiation present. By exposing the cells to a source of ionizing radiation, the cells are subjected to an increased amount of ionizing radiation when compared to the background. Hence, the cells are irradiated with a source of ionizing radiation which is non-natural, i.e. is above the background level. By irradiating the cells PARP is activated and PAR is synthesized. It is preferred to have PARP activated by ionizing radiation as it allows a consistent PARP activation in multiple samples simultaneously, e.g. in 96-wells format. This is because all the cells may easily be subjected to the same dose of ionizing radiation, as the distance and angle to the ionizing radiation source as well as the time of exposure of the cells can be controlled. PARP activation can thus be very well controlled by ionizing radiation. PARP activating compounds, i.e. DNA damaging agents, are reactive and need to diffuse to the cell nucleus to have their action. PARP activation may be less well controlled with a PARP activating compound as compared with irradiation. Nevertheless, it is envisioned that PARP may also be activated in the current invention by incubating the cells with a medium or buffer comprising a DNA damaging agent.

Hence, in one embodiment, PARP is activated by incubating the cells with medium comprising a PARP activating compound. A PARP activating compound may be an oxidizing agent, e.g. hydrogen peroxide or peroxynitrite, or a DNA-alkylating compound, e.g. N-methyl-N′-nitro-N-nitrosoguanidine (MNNG) or methyl methanesulphonate (MMS). In one embodiment, the PARP activating compound is selected from the group consisting of DNA alkylating compounds, oxidizing agents, hydrogen peroxide, peroxynitrite, N-methyl-N′-nitro-N-nitrosoguanidine (MNNG), methyl methanesulphonate (MMS), N-methylnitrosourea, triaziquonum, NO, calicheamicin gamma1, bleomycin, streptozotocin, etoposide and neocarzinostatin. In another embodiment, a PARP activating compound is selected from the group consisting of hydrogen peroxide, peroxynitrite, N-methyl-N′-nitro-N-nitrosoguanidine (MNNG), methyl methanesulphonate (MMS), N-methylnitrosourea, triaziquonum, NO, calicheamicin gamma1, bleomycin, streptozotocin, etoposide and neocarzinostatin. A PARP activating compound may also be a seen platinum compound, e.g. such as such as cisplatin, oxaliplatin. It is also possible to combine two or more of the different PARP activating compounds as listed above, i.e. to select at least 2, 3 or more PARP activating compounds from all of the PARP activating compounds, to be included in the medium to activate PARP.

These compounds are all DNA damaging agents and hence can activate PARP according to the invention. The medium according to this embodiment may be any medium, for example a medium which is used to culture the cells, the cells preferably being cells obtained from a subject. The medium may also be a buffer, e.g. PBS. In any case, the medium is compatible with the PARP activating compound and allows the compound to have contact with the cells such that the cells can take up the compound and the compound can have its action, i.e. activate PARP.

The method according to the invention also comprises a step of incubating the PARP activated cells prior to the PAR quantity determination step wherein the cells are incubated at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius.

When a PARP activating compound is used in the PARP activation step, it is understood that the subsequent incubation step as described below may include the PARP activating compound as well. This means that the subsequent incubation step can include PARP activation as well as it may be convenient e.g. to not exchange the medium. Hence, as such, the PARP activation step with a PARP activating compound and incubation step may be combined steps, i.e. having the single step of activating PARP and incubating the cells at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius.

The incubation in the presence of a PARP activating compound may also be carried out for some time, followed by e.g. exchanging the medium for a medium or buffer without the PARP activating compound such that two separate steps are performed, i.e. a PARP activation step and an incubation step. Alternatively, the PARP activation step may be carried out at a different temperature, e.g. at a temperature higher than the temperature of the incubation step. This may be because the PARP activating compound can be more active at a higher temperature, e.g. because of a higher diffusion rate. For example, PARP activation may be carried out with a PARP activating compound at a temperature of 37° Celsius, followed by incubation at a temperature below 37° Celsius, preferably below 25° Celsius, preferably below 20°, preferably at most or below 19°, 18°, 17°, 18°, 15°, 14°, 13°, 12°, 11°, 10°, 9°, 8°, 7°, 6°, 5°, 4°, 3°, 2°, or 1° Celsius, most preferably about 0° Celsius. The difference between the PARP activation step and incubation step being the temperature at which both steps are carried out. Similarly, when PARP activation involves irradiating the cells with a source of ionizing radiation, both the PARP activation step and incubation step may be combined in a single step. Hence it is understood that the PARP activation and incubation step can be separate steps or can involve the combination of both PARP activation and incubation in one step.

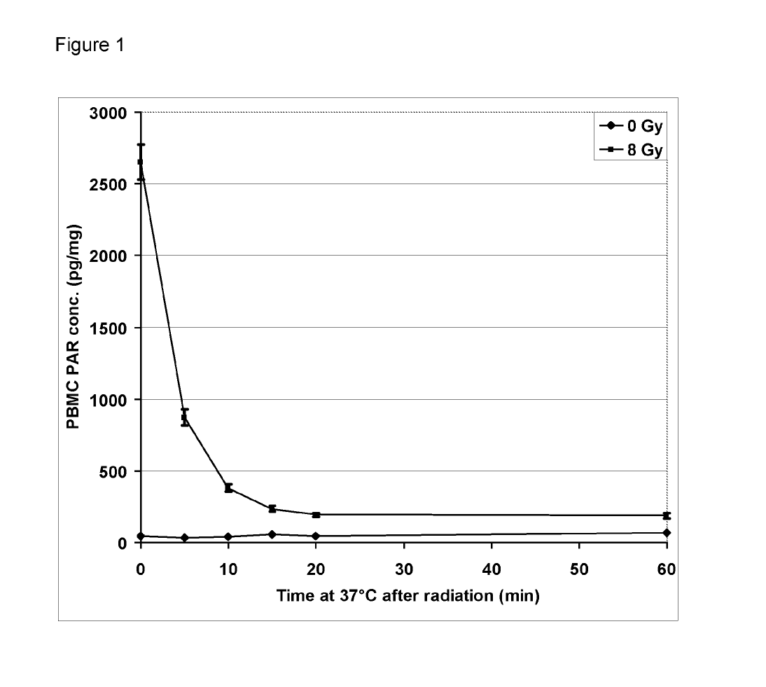

Hence, in one embodiment, the method of the invention is for determining PAR involving the steps of: providing cells; activating PARP in the cells and incubating the cells; determining the quantity of PAR in the PARP activated and incubated cells; wherein the cells are kept at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius. In one embodiment, in the method of the invention, the provided cells, the PARP activation and the incubation of the cells are all kept at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius. As can be observed from FIG. 1, incubating PARP activated cells at a temperature of 37° Celsius results in this example in a decrease of the PAR concentration from about 2500 pg/ml at the zero time point to about 200 pg/ml in about 20 minutes. It is to be noted that the cells were incubated on ice during the PARP activation step, i.e. subjecting the cells to ionizing radiation. Without being bound by theory, the activation of PARP induces the formation of PAR and other cellular activities such as by the PARG enzyme result in the degradation of PAR. When the temperature of the PARP activation step and/or incubation step is kept below 37° Celsius in accordance with the invention, the result is an increase of the PAR concentration (see e.g. FIG. 2A). Reducing the temperatures of the PARP activation step and/or incubation step gradually increases the PAR levels (FIG. 2B). Without being bound by theory, it is believed that by reducing the temperature of the incubation step (which as said may be combined with PARP activation) and optionally also of the PARP activation step, the activity of PARG is reduced, whereas the activity of PARP may substantially stay the same, resulting in a much higher and stable level of PAR in the cell when compared to cells irradiated and/or incubated at a higher temperature (compare e.g. FIGS. 1 and 2). FIG. 1 shows that the concentration of PAR reduces in time, whereas in FIG. 2 the PAR concentration of PAR increases in time, while in both scenarios steady state levels are achieved. Without being bound by theory, this indicates that the relative activity, i.e. PARP versus PARG is increased by incubating and optionally activating PARP at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius, i.e. the contribution of PARP activity has relatively become more resulting in more accumulation of PAR, which can be e.g. the result of a much lower PARG activity while the PARP activity is maintained. FIG. 2B shows that PAR induction and stability depends on the temperature of the incubation step. Without being bound by theory, this indicates that the relative activity of PARP and PARG is a reflection of the quantity of DNA lesions, hence the repair activity of the cells. It is understood that the incubating and/or irradiating step of the cells may not be performed at a temperature at which the cells are frozen, i.e. below the freezing temperature. With freezing temperature according to the invention is meant a temperature at which water in the buffer or medium in which the cells are kept turns into ice crystals that can disrupt or destroy the cell. With most buffer, e.g. PBS, or medium conditions in which cells are kept this is around 0° Celsius. Cells may also be kept at temperatures below 0° Celsius, e.g. −1° Celsius, −2° Celsius, −3° Celsius, −4° Celsius, −5° Celsius, −6° Celsius, −7° Celsius, −8° Celsius, −9° Celsius or even lower, e.g. by including in the buffer or medium a high concentration of carbohydrates or glycerol which are known to decrease the freezing point to temperatures below 0° Celsius. The incubating and/or irradiating step may be performed at a temperature as low as possible without allowing the cells to freeze, i.e. close to the freezing point. For example, the cells may be kept in a container which is kept in an ice bath which contains a mixture of ice and water. Hence, a lower limit with regard to temperature of the incubation and/or irradiation step may be about 0° Celsius. A lower limit with regard to temperature of the incubation and/or irradiation step may be above the freezing temperature.

The temperature of the incubation step and, optional PARP activation step does not necessarily have to be the same or have to be in the same range, although it may be preferred to have them at the same temperature or temperature range e.g. for convenience.

In one embodiment, the temperature of the PARP activation step is within the range from below 20° Celsius to above the freezing temperature. The temperature of the PARP activation step may also be within the range from below 20° Celsius to above −20° Celsius, from below 20° Celsius to −1° Celsius, −2° Celsius, −3° Celsius, −4° Celsius, −5° Celsius, −6° Celsius, −7° Celsius, −8° Celsius, or −9° Celsius. The temperature of the PARP activation step may also be within the range from below 20°, 19°, 18°, 17°, 16°, 15°, 14°, 13°, 12°, 11°, 10°, 9°, 8°, 7°, 6°, 5°, 4°, 3°, 2°, or 1° Celsius to the freezing temperature. The temperature of the PARP activation step may also be within the range from below 20°, 19°, 18°, 17°, 16°, 15°, 14°, 13°, 12°, 11°, 10°, 9°, 8°, 7°, 6°, 5°, 4°, 3°, 2°, or 1° Celsius to above −20° Celsius. The temperature of the PARP activation step may also be within the range from below 20°, 19°, 18°, 17°, 16°, 15°, 14°, 13°, 12°, 11°, 10°, 9°, 8°, 7°, 6°, 5°, 4°, 3°, 2°, or 1° Celsius to 0° Celsius.

In a particular embodiment, in a method according to the invention wherein the step of PARP activation involves irradiating the cells with a source of ionizing radiation, the step of PARP activation may also be carried at a temperature wherein the cells are frozen. As ionizing radiation can have its action on the DNA in cells in the frozen state, it is not necessarily required to have the cells above the freezing temperature during this step. This may be advantageous for example when cells have to be transported and/or stored before and/or after PARP activation. For example, cells may be quickly frozen on dry ice and for example shipped, also on dry ice, to a facility equipped for performing a method according to the invention involving the ionizing radiation step. Alternatively, the cells may also be transported for the irradiation step alone. Such a method may be in particular advantageous for cells obtained from a subject.

The step of PARP activation preferably is at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius. By incubating the cells for some time in a medium, e.g. a cold buffer, the level of PAR may reach a plateau. See for example FIGS. 1 and 2, which show a stabilization of the PAR level when comparing the 20 minutes and the 60 minutes time points, respectively. Hence, incubating the cells for some time may reduce the variability of the measurements between samples. Incubating cells at low temperatures (at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius.) may decrease a loss of PAR levels (FIG. 2B).

In one embodiment, in the method according to the invention the incubation period, wherein the temperature is kept below 37° Celsius preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius, is preferably at least 1 minute, preferably at least 2, 3, 4, or 5 minutes, preferably at least 10 minutes, preferably at least 15 minutes, preferably at least 30 minutes, most preferably at least 60 minutes. The upper limit of the incubation period may be at most 12 hours, 8 hours, preferably at most 6 hours. It is understood that the incubation period may relate to the incubation of the PARP activated cells. If according to the invention the PARP activation step and the incubation step are combined, the incubation period relates to the combined step of PARP activation and incubation.

Next, the quantity of PAR that is present in the cells is determined. Because in the cells PARP has been activated e.g. by irradiation with a source of gamma rays, and the cells have been incubated at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius the amount of PAR is increased compared to cells that are not subjected to the method of the invention. It was found that because the quantity of PAR is increased, the quantity of PAR can then be much more reliably determined, in particular in cells obtained from a subject. In addition, since the extent of PARP activation, e.g. by the same radiation dose, can vary from individual to individual (FIG. 6B), individual PARP activity induction values can be obtained.

As was already mentioned above, the methods of the invention are for determining PAR in cells, which cells are preferably cells obtained from a subject. As the level of PAR in cells is determined by the action of both the PARP and the PARG enzyme, the current invention now also allows for assessing quantitatively not only the effectiveness of PARP modulators, but the effectiveness of PARC modulators as well. Hence, the methods of the invention may also be used for assessing PARG activity, e.g. for assessing PARG modulators. For example, and in one embodiment, PARP is activated, and a first portion of cells is incubated at a temperature of about 37° Celsius and a second portion of cells is incubated at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius. Without being bound by theory, the activity of the PARG enzyme in the second portion of cells is now very low, allowing the synthesis and accumulation of PAR. In the first portion of cells incubated at a temperature of about 37° Celsius, the activity of the PARG enzyme is relatively high, resulting in the degradation of PAR. The difference between the determined PAR quantities is a measure of the PARG activity.

Alternatively, the cells are incubated after the low temperature incubation in a second incubation step, before the PAR quantity determination step, at a higher temperature, e.g. a temperature around 37° Celsius. When next the PAR quantity is determined, the PAR quantity may be taken as a measure for PARG activity. Hence, the methods of the invention for determining PAR as described herein may also be suitable for determining PARG activity. For example, and in one embodiment, the cells are incubated in the second incubation step in the presence of a PARG inhibitor and compared with another portion of cells from the same sample incubated in a second incubation step in the absence of a PARG inhibitor. In this method the effectiveness of a PARG inhibitor can be quantitatively assessed by comparing the PAR quantities determined under both conditions. The PARG inhibitor may be added to the provided cells, before or during the PARP activation step, or before or during incubation step at a low temperature, as long as the PARG inhibitor is added before the incubation at a higher temperature. It is understood that with a higher temperature according to the invention is meant that this temperature is relative to the temperature of the incubation step at a temperature below 37° Celsius according to the invention as discussed above. Preferably, the incubation with the PARG inhibitor is at a temperature of at least 20° Celsius, at least 25° Celsius, at least 30° Celsius, or at least 35° Celsius. The cells may be incubated at a temperature in the range of 20° Celsius to 43° Celsius. The incubation in the presence of a PARC inhibitor may be at a higher temperature of about 37° Celsius. Without being bound by theory, the higher the temperature, the more active the PARC enzyme will be, and conversely, the lower the temperature, the less active the PARG enzyme will be. Alternatively, and in another embodiment, the methods of the invention may also be used for screening of compounds for modulating PARC. Many alternative methods for quantitatively determining PARG activity and/or PARG inhibitors can be envisioned. For example, and in a further embodiment, the method may also include in addition to the presence or absence of a PARG inhibitor also the presence of a PARP inhibitor. This way, the PARC activity determination is less affected by newly generated PAR by the activity of the PARP enzyme. The PARP inhibitor may in this alternative embodiment be added after or during the PARP activation step, as long as PARP activation results in an increase of PAR to allow a proper quantitative assessment of PAR and hence of PARC activity. The second incubation step as indicated above may be at least 15 minutes, preferably at least 30 minutes, most preferably at least 60 minutes. The upper limit of the incubation period may be at most 12 hours, at most 8 hours, preferably at most 6 hours.

In another embodiment, PARP is activated, and cells are incubated at a temperature in the range of at least 20° C. and a second portion of cells is incubated at the same temperature. The second portion of the cells may be incubated in the presence of a compound assessed for PARG modulation activity, e.g. PARG activation or inhibition. Without being bound by theory, when the activity of the PARG enzyme in the second portion of cells is now very low, this will allow the synthesis and accumulation of PAR much more as compared to the first portion of cells incubated without the compound. Conversely, when the activity of the PARC enzyme in the second portion of cells is now very high, this will inhibit the synthesis and accumulation of PAR as compared with the first portion of cells incubated without the compound. Hence, the difference between the determined PAR quantities is a measure of the PARG activity, and hence allows to assess compounds with regard to PARG modulatory activity. Off course, as PAR levels are also determined by PARP activity, additional control experiments may be carried out to exclude effects of identified compounds on PARP activity. Also, the screening method may also include the addition of a known PARP inhibitor to exclude possible effects of the compounds that are screened on PARP activity. For example, first PARP activated cells may be incubated for some time to allow PAR accumulation. Next, both the compound assessed for PARC modulation activity and the known PARP inhibitor may be added to the cells simultaneously.

Hence, in one embodiment a method is provided for screening compounds for modulating PARC comprising the steps of:
providing cells ex vivo;
activating PARP in the cells;
incubating the PARP activated cells;
determining the quantity of PAR in the incubated PARP activated cells;

wherein of the provided cells that are incubated, a first portion is incubated in the presence of a compound, and a second portion is incubated in the absence of the compound, and wherein the incubation in the presence or absence of the compound is carried out a temperature of at least 20° C., and wherein the PAR quantities are subsequently independently determined for the first and second portion, and comprising the step of comparing the determined quantities of PAR. A compound modulating PARG may be identified when in the comparison the determined PAR quantities are different. For example, and as already explained above, when the amount of PAR determined is increased compared to the control, a compound may have inhibited PARC, and when the amount of PAR determined has decreased compared to the control, a compound may have promoted PARC activity. Also provided are kits for determining PARC activity, the kit comprising written instructions to perform any of the steps of the methods according the invention such as described above and below for screening compounds for modulating PARG.

In one embodiment, a method is provided for screening compounds for modulating PARC comprising the steps of:

providing cells ex vivo;

activating PARP in the cells;

incubating the PARP activated cells in the presence of a compound;

determining the quantity of PAR in the incubated PARP activated cells;

wherein the incubation in the presence or absence of the compound is carried out a temperature of at least 20° C., and wherein the PAR quantity is subsequently compared with a reference value. It is understood that the reference value may be from cells that were incubated in the presence of a compound known to e.g. inhibit PARG, when the PAR quantity is in a similar range, the compound may be identified as being a PARG inhibitor.

The steps of the methods for screening compounds for modulating PARG may also be used for determining the suitability and/or dosing of a PARC inhibitor for the treatment of a subject and/or determining the dosing of radiation therapy for the treatment of a subject, similar to as is described for PARP inhibitors herein.

As already indicated above, the methods of the invention are for determining PAR in cells, which is a measure of PARP activity and/or PARC activity in the cells. The methods of the invention thus may also be referred to as methods for determining PARP activity and/or PARG activity in cells. The methods of the invention may also be referred to as methods for use in screening for compounds for modulating PARP or PARC activity.

In one embodiment, the PAR quantity determination step comprises a step of lysing of the cells and a step of determining the quantity of PAR with an ELISA. Many methods are available for lysing cells. Lysing the cells may involve freezing and thawing, mechanical homogenization, sonification and may involve the use of lysis buffers comprising detergents such as deoxycholic acid, triton, SDS, and using enzymes to degrade RNA/DNA in order to reduce viscosity of the lysate. When the cells are lysed, the amount of PAR that is formed can remain stable. When the lysate is stored at a lower temperature, for example at 16° Celsius, at 4° Celsius or 0° Celsius, the PAR quantity levels will remain even more stable, and this is thus preferred. Freezing the cell lysate, e.g. at −20° Celsius or −80° Celsius allows to maintain the PAR levels in the cellular lysate for longer periods (see FIG. 7). The cells may also be pelleted and the cell pellets frozen, the cells may subsequently be thawed and subjected to lysis, e.g. by suspending the cell pellet in lysis buffer at the time the samples are to be analyzed.

Alternative PAR quantity determination steps may comprise FACS analysis, western blotting, dot blotting which methods also involve antibodies against PAR. Other methods that do not involve antibodies may also be envisaged such as the determination of the amount of incorporated biotinylated or radioactive NAD in PAR.

In one embodiment, the cells obtained from a subject are mammalian cells, preferably human cells. Preferably the cells obtained from a subject are from a tissue biopsy or are from peripheral blood mononuclear cells. A tissue biopsy can be obtained from diseased tissue, e.g. cancer tissue. A tissue biopsy can also be obtained from healthy tissue. From a single patient, tissue biopsies can be provided from both diseased and healthy tissue. This is for example useful for comparing PARP activity and/or effects of ionizing radiation in these tissues in order to predict effects of a treatment with a PARP inhibitor and/or radiation therapy. These types of cells are suitable for the assay.

In another embodiment, in a method according to the invention, cells are provided, optionally mildly lysed, optionally stored and/or optionally frozen prior to the PARP activation step while preserving PARP activity. Preserving PARP activity may include freezing, storing and/or mild lysing. Mild lysis according to this embodiment involves not the complete disintegration of cells, but involves lysing to such an extent that the cell nucleus remains substantially intact. The mild lysing of this embodiment is different from the lysis that is carried out when the cells are lysed for determining the quantity of PAR in the PARP activated cells such as described in the examples in item 5 relating to the preparation of cell lysate. Suitable buffers that can provide for a mild lysis are e.g. a buffer with 20 mM HEPES-KOH, 5 mM KCl, 1.5 mM MgCl2 and a pH of 8.0. Having the cell nucleus remaining substantially intact according to this embodiment includes having the cell nucleus with a partly permeable cell nucleus. By having a mild lysis according to this embodiment, PARP and/or PARG can remain in the proximity of the DNA. Buffers used to preserve PARP activity may contain glycerol, salts and buffers to set physiological pH conditions. Preserving PARP activity may include preservation of PARG activity as well. Mildly lysing the cells accordingly may for example be advantageous when the cells are frozen and stabilized without irreversibly impairing PARP activity and/or PARG activity, because the samples may be transported or stored for some time before carrying out the method of the invention in a similar fashion as when compared to the method carried out on live cells. The method of this embodiment may be in particular useful for carrying out a method according to the invention in which PARP is activated by irradiating the cells with a source of ionizing radiation. As said, the PARP activation step may also be carried out while having the cells, or lysed cells according to this embodiment, frozen. This may be advantageous as not every laboratory may have access to a source of ionizing radiation and thus this method may allow for transport of samples such that the method may be carried out off site.

In another aspect of the invention, method for determining PAR in cells may comprise the step of: providing cells; irradiating the cells with a source of ionizing radiation; and determining the quantity of PAR in the irradiated cells. Hence, in this embodiment, according to a different aspect of the invention, the cells do not need to be incubated in a separate step. During the step of irradiating the cells with a source of ionizing radiation, the cells are preferably kept at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius. In one embodiment, in a method according to the invention the PARP activation step comprises irradiating the cells with a source of ionizing radiation.

In the irradiation step, i.e. the PARP activation step wherein the cells are irradiated with a source of ionizing radiation, the cells are exposed to a radiation dose that exceeds background levels. The dose of ionizing radiation preferably is at least 0.2 Gray. The dose of ionizing radiation preferably is at least 0.2 Gray as applied by the Gammacell® 40 Exactor as described in the examples. More preferably, it is at least 3 Gray, most preferably at least 8 Gray, as applied by the Gammacell® 40 Exactor as described in the examples. More preferably, it is at least 3 Gray, most preferably at least 8 Gray. More preferably, it is at least 3 Gray, most preferably at least 8 Gray, as applied by the Gammacell® 40 Exactor as described in the examples. At these doses there is a linear correlation between radiation dose and the amount of PAR induction. The maximum radiation dose is the maximum radiation dose with which there still is a linear correlation in between the radiation dose and the amount of PAR induction. It is well understood that when the radiation dose exceeds the maximum dose there will no longer be a linear correlation between radiation dose and the amount of PAR induction. This can occur for example when NAD+ is depleted and becomes a limiting factor. Hence, the maximum radiation dose may depend on factors such as NAD+ concentration, energetic condition of the cells, and the cell type. The skilled person can easily determine the maximum dose by performing a dose response curve on a representative sample of cells and by determining the concentration at which the dose response curve starts deviating from the linear correlation (see FIG. 3 which shows the linear correlation). A maximum dose that can be selected can be for example at most 16 Gray.

As said, sources of ionizing radiation such as gamma rays are widely known, e.g. cesium-137, iridium-192 and cobalt-60. As long as the cells can be exposed to an appropriate dose of ionizing radiation, i.e. an amount of ionizing radiation in a relatively short time interval, that is sufficient to activate PARP, such a dose and radiation source can be used in the invention. The Gray (Gy) is a unit used to measure the quantity of absorbed dose and relates to the amount of energy actually absorbed, and is used for any type of radiation and any material. One gray is equal to one joule of energy deposited in one kg of a material. Absorbed dose is often expressed in terms of hundredths of a gray, or centi-grays. One gray is equivalent to 100 rads. For example, and as outlined in the examples, a Gammacell Exactor 40 device may be used to provide for the above disclosed doses of gamma rays. The device can be obtained from Best Theratronics, Ottawa, Canada. This device comprises a Cesium-137 gamma ray source, which emits a central dose of 1.1 Gy/minutes. Hence, administering a dose of 8 Gy using this device would require about 8 minutes of exposure.

In one embodiment, in a method according to the invention, of the provided cells, a first portion of the provided cells is subjected to PARP activation and a second portion of the provided cells is not subjected to PARP activation, and wherein the PAR quantities are subsequently determined independently for the first and second portions, and comprising the step of comparing the determined quantities of PAR. In this embodiment, the effect of activating PARP, e.g. irradiating the cells from a subject with a source of ionizing radiation, can be monitored.

In another embodiment, a method is provided for screening compounds for modulating PARP, comprising an additional cell culturing step before the PARP activation step, wherein of the provided cells, a first portion of the provided cells is cultured in the presence of a compound, and a second portion of the provided cells is cultured in the absence of a compound, and wherein the PAR quantities are subsequently independently determined for the first and second portions, and comprising the step of comparing the determined quantities of PAR. In this embodiment, the effect of a compound on PARP can be monitored in cells, preferably in cells obtained from a subject. It is understood that culturing according to this embodiment may also involve incubating. In a further embodiment, the compound is a compound screened for inhibiting PARP. It is understood that the compound may also be a compound being screened for activating PARP, i.e. has the effect of increasing the activity of PARP resulting in an increase of PAR being produced. This increase of PAR is in addition and separate from the PARP activation step. In this embodiment, libraries of compounds may be screened for PARP activation and/or PARP inhibition.

In one embodiment, a method according to the invention comprises providing cells obtained from a subject obtained at different time points, and wherein for each time point the PAR quantity is determined thereby determining PAR in cells from a subject in time. This way PARP activity may be monitored in time in a subject.

In one embodiment, a method according to the invention comprises providing cells obtained from a subject obtained at different time points, and wherein for each time point the PAR quantity is determined thereby determining PAR in cells from a subject in time, and wherein the different time points comprise at least two time points, wherein at a first time point, a subject has not been treated with a PARP inhibitor and wherein at a second time point, the subject has been treated with a PARP inhibitor, and wherein determined PAR quantities from at least the first and the second time points, preferably all time points, are compared. In this embodiment, the effect of a PARP inhibitor may be monitored. In an alternative embodiment, the method of the inventions are for determining the dosing of radiation therapy for the treatment of a subject, wherein the provided cells are obtained from a subject and wherein based on the determined PAR quantity the dosing of radiation therapy is determined. The PAR level may correlate with the damage in the cells. A reduction in PAR with time reflects the cellular repair activity on the PAR inducing radiation lesions of the analysed cells. Lesion induction and repair values (reduction within a certain time period), as measured by radiation induced PAR levels, on patient derived cells can therefore predict inter-individual cellular sensitivity to radiation. Radiotherapy induced tissue toxicity (patient sensitivity to radiation) is also determined by the strength of the inflammatory and fibrotic response upon damage (radiation). PARP activation drives this damage induced response cascade and inhibition reduces inflammatory parameters and ultimately toxicity. The inter-individual variation in the PARP activation capacity can therefore correlate with radiation induced normal tissue toxicity (inflammation/fibrosis). The level of PARP activation, either by a PARP activating compound or irradiation, can be a measure for the sensitivity to radiation therapy. Preferably, the PARP activation step of this embodiment comprises irradiating the cells with a source of ionizing radiation according to the invention.

The PAR quantities from the provided cells obtained from a subject obtained at different time points, may be determined as soon as the cells are provided. Hence, the PAR quantities as determined may be derived from different assays, i.e. performed on different moments in time. Also, as already mentioned, cells obtained from subjects may be stored under appropriate conditions such that all cells can be collected and PAR determined in a single assay. Alternatively, the cells that are preferably obtained from subjects, may be processed, i.e. performing the steps according to the invention of PARP activation and incubation, at a temperature below 37° Celsius, preferably below 25° Celsius, preferably below 20°, preferably at most or below 19°, 18°, 17°, 16°, 15°, 14°, 13°, 12°, 11°, 10°, 9°, 8°, 7°, 6°, 5°, 4°, 3°, 2°, or 1° Celsius, most preferably about 0° Celsius and subsequently lysing the cells to obtain a lysate. This lysate may be stored e.g. by freezing the lysates. Different lysates may be obtained from different time points and when obtained may be subjected to a single assay for determining PAR, e.g. an ELISA such as described below.

In one embodiment, in a method according to the invention, a method is provided for determining the dosing of radiation therapy for the treatment of a subject, wherein the provided cells are obtained from a subject, and wherein based on the determined PAR quantity the dosing of radiation therapy is determined. The PARP activation step in this method does not necessarily have to be an irradiation step. However, an irradiation step is preferred. By determining the response to PARP activation, it may be predicted how a subject will respond to radiation therapy, and the radiation dose may be determined based on the amount of PARP activation, e.g. relative amount of PAR produced in cells from a subject.

In another embodiment, the methods of the invention are for determining the suitability and/or dosing of a PARP inhibitor for the treatment of a subject and/or for determining the dosing of radiation therapy for the treatment of a subject, comprising an additional step of culturing a portion of the cells in the presence of a PARP inhibitor before the PARP activation step and wherein based on the determined PAR quantity the suitability and/or dosing of a PARP inhibitor is determined and/or dosing of irradiation is determined for the treatment of a patient. In this embodiment, the effect of a PARP inhibitor can be determined for a subject, and the effect of a radiation therapy may be determined as well. Hence, in this embodiment, dependent on the outcome of the PAR levels under the different conditions, the suitability and/or dosing of a PARP inhibitor may be selected and/or the radiation dose may be selected as well. Patients may differ with regard to PARP induction when exposed to irradiation and/or may differ with regard to the effect of PARP inhibitors. By testing cells from a subject ex vivo before and/or during therapy, e.g. radiation therapy, PARP inhibitor treatment or both radiation therapy and PARP inhibitor treatment combined, an optimal therapy may be selected for the subject. The cells may be PBMCs obtained from the blood of subjects. Cells may also be cells obtained from diseased tissue, e.g. a tumour biopsy. Also, it is envisioned that the methods of the invention can be used on cells obtained from a subject from non-diseased tissue and cells from diseased tissue and that the comparison of the quantities of PAR produced in the cells of the different tissues allows determining an optimal treatment with radiation and/or PARP inhibitor.

In another embodiment, the determination step for the suitability and/or dosing of a PARP inhibitor and/or determination step for the dosing of radiation therapy, for the treatment of a subject comprises comparing the determined PAR quantity with a reference value. For example, reference values of PAR may represent a group of patients responding well to a certain dose of PARP inhibitor or a certain dose of radiation. PAR levels measured above or below the reference value may determine the suitability of the subject for the PARP inhibitor and/or radiation therapy.

In one embodiment, multiple portions from the provided cells obtained from a subject are subjected to different doses of irradiation or different doses of PARP activating compounds and/or different concentrations of PARP inhibitor, and wherein for each portion the PAR quantity is determined.

When cells obtained from a subject are subjected to irradiation only and the value of the intracellular PAR quantity of the patient cells is increased relative to a reference value (determined e.g. from population wide studies), this may indicate that this subject responds strongly to radiation treatment and might have an increased risk to develop radiation induced toxicities, hence tolerates the treatment less. A lower radiation dose or exclusion from combination treatment may be considered. Conversely, when the intracellular radiation induced PAR quantity of the subject cells is decreased relative to a reference value, this may indicate that the subject shows a lower response, thus may be subjected to a higher dose of radiation treatment.

In another embodiment, from the same subject, cancer cells and/or healthy cells are obtained and both types of obtained cells are subjected to a suitability determination method described above in separate assays and a relative PAR quantity is determined (e.g. PAR value in cancer cells divided by PAR value in healthy cells) and these are compared to a reference value.

Hence, in the methods of these embodiments, cells obtained from a subject may be divided in multiple portions, and each portion may be subjected to e.g. different doses of irradiation and/or different concentrations of a PAR level modulating compound, e.g. a PARP inhibitor, and for each portion the PAR quantity is determined. In the simplest scenario, a first portion of the cells is subjected to a first radiation dose and a second portion is not subjected to a radiation dose. See for example FIG. 6A/B. The same accounts for the PARP inhibitor, a first portion of the cells is subjected to a first concentration of the PARP inhibitor and a second portion is not subjected to the PARP inhibitor (see for example FIG. 4, the 0 nM point from the curve compared with any of the other points). The two values may be used to determine a ratio which can be used in the selection and/or the suitability step. The ratio may also be compared with a reference value. When many more portions are subjected to different conditions, a dose response curve may be provided for each patient based on which the radiation dose is selected and/or suitability of the PARP inhibitor is determined. For example, in FIG. 4 a dose response curve for Olaparib is shown for a particular patient that may be suitable for treatment with standard Olaparib and/or standard radiation therapy. The FIG. 4 curve may be regarded as a reference curve. When the dose response curve would be shifted to the left, PARP inhibition, i.e. by Olaparib, efficiency is greater in this particular patient and the dose of Olaparib may be reduced. Conversely, the Olaparib dose may remain unchanged and the radiation dose may be reduced when compared to the standard radiation therapy. Should the dose response curve shift to the right, the Olaparib dose and/or radiation dose may be increased for that particular patient or a different compound with a better activity determined and tested similarly may be selected.

In one embodiment, the PAR quantification, e.g. ELISA, comprises the steps of:

contacting the cellular lysate with a first antibody which can be an immobilized monoclonal antibody capable of binding PAR;

contacting PAR bound to the first monoclonal antibody with a second antibody which is a polyclonal antibody capable of binding PAR;

subsequently detecting the quantity of second antibody bound to PAR thereby determining the quantity of PAR; and

wherein the ELISA comprises after a contacting step one or more washing steps.

Furthermore, detecting the quantity of second antibody can comprise the steps of:

contacting the second antibody, bound to PAR bound to the first antibody, with a third antibody which is an antibody capable of binding the polyclonal antibody and which is not capable of binding the monoclonal antibody, and wherein the third antibody is conjugated to an enzyme

providing a substrate for the conjugated enzyme;

contacting the conjugated enzyme with the substrate and allowing the conversion of the substrate;

measuring the conversion of substrate thereby detecting the quantity of second antibody bound to PAR.

The enzyme which is conjugated to the third antibody may be horse radish peroxidase. Substrates that may be used are widely commercially available and can be chromogenic substrates or chemiluminescent substrates. For example, substrates may be selected from the group consisting of TMB (3,3′,5,5′-tetramethylbenzidine), DAB (3,3′-diaminobenzidine), ABTS (2,2′-azino-bis(3-ethylbenzothiazoline-6-sulphonic acid). Other commercially available substrates are for example the PeroxyGlow substrate from Trevigen®.

### EXAMPLES

The PARP activity assay protocol was performed in accordance with the Trevigen® HT (High Throughput) PARP in vivo Pharmacodynamic Assay II (Trevigen® catalogue number 4520-096K), which is incorporated herein by reference. The assay was used to quantify the amount of PAR in peripheral blood mononuclear cells (PBMC).

The protocol below has been modified by including an embodiment of the incubation step and/or irradiation step according to the invention.

Materials Supplied with the Kit

| Catalog | | amount | | |
|---|---|---|---|---|
| Number | Component | Provided | | |
| | | | | Storage T^{a} |
| 4520-096-01 | PAR Standard | 5 | pg/μl | −80° C. |
| | | 5 × 20 | μl | |
| 4520-096-02 | Sample Buffer | 0 | ml | 4° C. |
| 4520-096-03 | PAR Detecting | 30 | μl | −20° C. |
| | PolyclonalAntibody | | | |
| 4520-096-04 | Goat anti-Rabbit IgG-HRP | 30 | μl | −20° C. |
| 4675-096-01 | PARP PeroxyGlow ™ A | 6 | ml | 4° C. |
| 4675-096-02 | PARP PeroxyGlow ™ B | 6 | ml | 4° C. |
| 4520-096-05 | Cell Lysis Reagent | 30 | ml | 4° C. |
| | | | | Storage T |
| 4520-096-06 | DNase I, 2 Units/μl | 60 | μl | −20° C. |
| 4520-096-07 | 100X Magnesium Cation | 500 | μl | 4° C. |
| 4520-096-P | Pre-coated white 96-stripwell | 1 | plate | 4° C. |
| | plate, and 5 sealers | | | |
| 4520-096-08 | Jurkat Cell Lysate Standard | 600 | μl | −80° C. |
| | Control, Low | | | |
| 4520-096-09 | Jurkat Cell Lysate Standard | 600 | μl | −80° C. |
| | Control, Medium | | | |
| 4520-096-10 | Jurkat Cell Lysate Standard | 600 | μl | −80° C. |
| | Control, High | | | |
| 4520-096-11 | Antibody Diluent | 15 | ml | 4° C. |
| 4520-096-12 | 20% (w/v) SDS | 1 | ml | RT |

^{a}° C. = 4° Celsius)

Materials Prepared which are not Included in the Trevigen® Kit 4520-096K

| Cat# | Component | supplier |
|---|---|---|
| 04693159001 | Complete-EDTA free | Roche |
| | PBS sterile 500 ml | Invitrogen |
| 23227 | BCA protein assay | Thermo Scientific |
| 78830 | PMSF | Fluka |
| 14190 | Phosphate buffered saline (PBS) | Gibco |
| 362782 | BD vacutainer ® CPTTM | Becton Dickinson |

### Reagent Preparation

PBST: Add 500 μl Tween-20 to 500 ml sterile PBS, mix well and store at 4° C.

Cell lysis buffer: Transfer 30 ml cell lysis reagent (cat#4520-096-05) to a 50 ml tube, add 5.23 mg PMSF (1 mM end concentration), add three Complete EDTA free tablets and sonicate for 15 minutes at room temperature. Centrifuge at 3000×g for 10 minutes, aliquot and store at −20° C.

### 1. Preparation of PBMCs or Cells

Peripheral Blood Mononuclear Cells (PBMCs) were prepared by drawing 8 ml blood from a subject in a BD vacutainer CPT-citrate tube (RT) at the clinic. Invert the tubes three times to mix the anti-coagulant (1 ml) with the blood and keep the tube at room temperature (RT). Centrifuge for 25 minutes at 1500×g at RT, and invert the tubes three times. Pour the upper layer into a 50 ml Falcon tube on ice and incubate on ice for 5 minutes. Adjust the total volume to 50 ml with ice-cold PBS and centrifuge 10 minutes at 330×g at 4° Celsius. Decant the supernatant and place the tube inverted on filter paper for 2 seconds. Re-suspend the pellet in 10 ml ice-cold PBS. Count the PBMCs with a hema-cytometer and centrifuge 10 minutes at 330×g at 4° Celsius. Re-suspend the pellet at 2×10exp6 PBMC/500 μl in PBS and transfer to a pre-chilled 2.0 ml tube on ice.

Alternatively, cultured cells, such as cell lines, are prepared by seeding cells onto 6 cm dishes. Culture adherent cells until 60-80% confluence (2-6×10E6 cells). Steps 2-4 can be performed with cells grown on the dish or alternatively after collection by trypsinisation or scraping, centrifugation and re-suspension in medium to an appropriate cell density.

### 2. Exposure to Inhibitors (Optional)

Next, the cells can optionally be incubated in the presence or absence of a PARP or PARG inhibitor (or a compound screened for such PARP or PARG inhibition) before the subsequent PARP activation step, such as an irradiation step. Cells are suspended at 2×10E6 PBMC/500 μl culture medium (without fetal calf serum) and incubated with a inhibitor (for example for PARP or PARG) at e.g. 37° Celsius for about 1 h. Subsequently samples are chilled for 5 minutes on ice.

Alternatively, cultured cells, can be incubated with inhibitors for an appropriate time while in culture on a dish or upon collection by trypsinisation or scraping, centrifugation in re-suspension in medium. Subsequently samples are chilled for 5 minutes on ice.

### 3. PARP Activation

Irradiate cells using a Cesium-137 source with a Gammacell® 40 Exactor (Best Theratronics, Ottawa, Canada) according the manufacturer's instructions. The samples can be irradiated at different doses, and/or at different temperatures, as indicated in the figures and results. For example, the samples are irradiated in a horizontal row of 2 ml eppendorf tubes covered with ice in a box.

Alternatively, cells can be exposed to DNA damaging agents such as hydrogen peroxide (H₂O₂). For example, cells are suspended at 2×10E6 PBMC/500 μl culture PBS. H₂O₂ in PBS can be added to the cells to a final concentration of for example 10 or 100 μM (micro molar). The samples can be treated at different doses as indicated in FIG. 2B. Cells are mixed by shaking or vortexing. Samples are incubated for at least 5 minutes on ice (step 4) and can be (optionally) centrifuged and re-suspended in medium thereafter.

### 4. Incubation at Low Temperature

After the PARP activation step, the activated cells can be incubated for an appropriate time period at different temperatures, as shown in the results. For example, after the irradiation step, the irradiated cells are incubated for 1 hour on ice. Alternatively, after the treatment with H₂O₂, cells can be incubated for an appropriate time period at different temperatures. For example, the cells can be incubated for 1 hour on ice. H₂O₂ can be maintained in the buffer or medium during this incubation step.

### 5. Preparation of Cell Lysate

Next, the cells are centrifuged for 20 seconds at 10000×g at 4° Celsius and the supernatant aspirated. PBMCs are re-suspended in 100 μl ice-cold Cell lysis buffer (2×10exp7 PBMC/ml) and vortexed well prior incubation on ice for 15 minutes with periodically vortexing. SDS is added to a final concentration of 1% and the suspension is mixed by vortexing. The cell extract is incubated at 100° Celsius for 5 minutes and transferred on ice. 1 μl of “Mg Cation” solution is added and 0.4 μl of DNase I for each sample of 100 μl. Vortex briefly at setting 50% and incubate at 37° Celsius for 90 minutes. Aliquots are taken to determine the protein concentration and for further analysis. The samples are diluted at least three times with sample buffer (Trevigen® HT PARP in vivo Pharmacodynamic Assay II) and the remaining undiluted extract is stored at −80° Celsius.

Alternatively, when using adherent cultured cells that are treated (step 1-4) on the dish the medium is removed from the cells next. The cells are washed with ice-cold PBS. 500 μl of ice-cold Cell lysis buffer (Trevigen® HT PARP in vivo Pharmacodynamic Assay II) is added to the cells. Immediately thereafter, the cells are scraped with a cell scraper to detach them from the culture dish. The cells are incubated in the Cell lysis buffer on ice with periodically scraping. The cell suspensions are transferred into a 2.0 ml tube on ice. SDS is added to a final concentration of 1% and the suspension is mixed by vortexing. The cell extract is incubated at 100° Celcius for 5 minutes and transferred on ice. 1 μl of “Mg Cation” solution and 0.4 μl of DNase I are added to each sample of 500 μl. Vortex briefly and incubate at 37° Celcius for 90 minutes. Aliquots are taken to determine the protein concentration and for further analysis. The samples are diluted at least three times with sample buffer (Trevigen® HT PARP in vivo Pharmacodynamic Assay II) and the remaining undiluted extract is stored at −80° Celcius.

### 6. Prepare PAR Dilutions for the Standard Curve

One vial of the PAR standard is taken (20 μl at 25.000 pg/ml) from −80° Celsius and thawed on ice, spun down, vortexed and mixed. A serial dilution is made with the supplied standard stock containing 25.000 pg/ml PAR. A PAR concentration range from 10 pg/ml to 1000 pg/ml is prepared on ice as follows:

| | conc. pg/ml | PAR standard (μl) | sample buffer (μl) |
|---|---|---|---|
| a | 1000 | 16 (provided PAR standard) | 384 |
| b | 500 | 200 (of a) | 200 |
| c | 200 | 160 (of b) | 240 |
| d | 100 | 200 (of c) | 200 |
| e | 50 | 200 (of d) | 200 |
| f | 20 | 160 (of e) | 240 |
| g | 10 | 200 (of f) | 200 |
| h | 0 | 0 | 200 |

### 7. ELISA Trevigen® Preparation and PAR Quantification

Remove the needed number of pre-coated strip wells from the foil pouch. Add 50 μl of each of the 8 standard dilutions a-h (step 6) to the provided Trevigen® pre-coated white 96-stripwells in triplo and seal. Dilute the lysates as indicated above (step 5) and pipet 50 μl of sample into each well in triplo. Seal the wells and incubate overnight at 4° Celsius. Dilute the PAR Detecting Polyclonal Antibody (provided in the kit) 500-fold in Antibody Diluent and incubate at 37° Celsius for 1 h. Wash the wells 4 times with 300 μl PBST and remove all liquid. Add 50 μl of diluted PAR Detecting Polyclonal Antibody to each well and incubate 2 h at room temperature. Wash the wells 4 times with 300 μl PBST and remove all liquid. Dilute Goat anti-Rabbit IgG-HRP secondary antibody (provided in kit) 250-fold in ice-cold Antibody Diluent and incubate at 37° Celsius for 1 h. Add 50 μl of diluted HRP antibody to each well and incubate 1 h at room temperature in the dark. Wash the wells 4 times with 300 μl PBST each time. Remove all liquid and cover with a seal and aluminum foil. Measure the plate with a 96 well plate reader able to measure chemi-luminescence.

Results from different experiments are depicted in FIGS. 1-8.

1. A method for determining PAR in cells ex vivo comprising the steps of:
providing cells ex vivo;
activating PARP in the cells;
incubating the PARP activated cells;
determining the quantity of PAR in the incubated PARP activated cells;
wherein the cells are kept at a temperature below 20° Celsius during the incubation step and optionally during the PARP activation step.

2. Method according to claim 1, wherein the cells are kept at a temperature, during the incubation step and optionally during the PARP activation step, below 15° Celsius, preferably at most 10° Celsius, preferably below 4° Celsius, most preferably about 0° Celsius.

3. Method according to claim 1, wherein the incubation period is at least 5 minutes, preferably at least 10 minutes, preferably at least 30 minutes, most preferably at least 60 minutes.

4. Method according to claim 1, wherein PARP is activated by incubating the cells with a PARP activating compound.

5. Method according claim 4, wherein the PARP activating compound is selected from the group consisting of DNA alkylating compounds, oxidizing agents, hydrogen peroxide, peroxynitrite, N-methyl-N′-nitro-N-nitrosoguanidine (MNNG), methyl methanesulphonate (MMS), N-methylnitrosourea, triaziquonum, NO, calicheamicin gamma1, bleomycin, streptozotocin, etoposide and neocarzinostatin.

6. Method according to claim 1, wherein PARP is activated by irradiating the cells with a source of ionizing radiation.

7. Method according to claim 6, wherein the irradiation step comprises a radiation dose of at least 0.2 Gray, preferably at least 3 Gray, most preferably at least 8 Gray.

8. Method according to claim 1, wherein the PAR quantity determination step comprises a step of lysing of the cells and a step of determining the quantity of PAR with an ELISA.

9. Method according to claim 1, wherein the provided cells are obtained from a subject.

10. Method according to claim 9, wherein the subject is a mammal, preferably a human.

11. Method according to claim 10 wherein the provided cells obtained from a subject are from a tissue biopsy or are peripheral blood mononuclear cells.

12. Method according to claim 11, wherein of the provided cells, a first portion of the provided cells is subjected to PARP activation and wherein a second portion of the provided cells is not subjected to PARP activation, and wherein the PAR quantities are subsequently determined independently for the first and second portions, and comprising the step of comparing the determined quantities of PAR.

13. Method according to claim 1, for screening compounds for modulating PARP, comprising an additional cell culturing step before the PARP activation step, wherein of the provided cells, a first portion of the provided cells is cultured in the presence of a compound, and a second portion of the provided cells is cultured in the absence of a compound, and wherein the PAR quantities are subsequently independently determined for the first and second portions, and comprising the step of comparing the determined quantities of PAR.

14. Method according to claim 13, wherein the screening is for compounds inhibiting PARP.

15. Method according to claim 1, comprising providing cells obtained from a subject obtained at different time points, and wherein for each time point the PAR
quantity is determined thereby determining PAR in cells from a subject in time.

16. Method according to claim 15, wherein the different time points comprise at least two time points, wherein at a first time point, a subject has not been treated with a PARP inhibitor and wherein at a second time point, the subject has been treated with a PARP inhibitor, and wherein determined PAR quantities from at least the first and the second time points, preferably all time points, are compared.

17. Method according to claim 8, for determining the dosing of radiation therapy for the treatment of a subject, wherein the provided cells are obtained from the subject and wherein based on the determined PAR quantity the dosing of radiation therapy is determined.

18. Method according to claim 9, for determining the suitability and/or dosing of a PARP inhibitor for the treatment of a subject and/or for determining the dosing of radiation therapy for the treatment of a subject, comprising an additional step of culturing a portion of the cells in the presence of a PARP inhibitor before the PARP activation step and wherein based on the determined PAR quantity the suitability and/or dosing of a PARP inhibitor is determined and/or dosing of irradiation is determined for the treatment of a patient.

19. Method according to claim 17, wherein the determination step for the dosing of a PARP inhibitor and/or determination step for the dosing of radiation therapy, for the treatment of a subject comprises comparing the determined PAR quantity with a reference value.

20. Method according to claim 14, wherein multiple portions from the provided cells obtained from a subject are subjected to different doses of irradiation or different doses of PARP activating compounds and/or different concentrations of PARP inhibitor, and wherein for each portion the PAR quantity is determined.

21. A method for screening compounds for modulating PARG comprising the steps of:
providing cells ex vivo;
activating PARP in the cells;
incubating the PARP activated cells;
determining the quantity of PAR in the incubated PARP activated cells;
wherein of the provided cells that are incubated, a first portion is incubated in the presence of a compound, and a second portion is incubated in the absence of the compound, and wherein the incubation in the presence or absence of the compound is carried out a temperature of at least 20° C., and wherein the PAR quantities are subsequently independently determined for the first and second portion, and comprising the step of comparing the determined quantities of PAR.

22. A method according to claim 8, wherein the ELISA comprises the steps of:
contacting the cellular lysate with a first antibody which is an immobilized monoclonal antibody capable of binding PAR;
contacting PAR bound to the first monoclonal antibody with a second antibody which is a polyclonal antibody capable of binding PAR;
subsequently detecting the quantity of second antibody bound to PAR thereby determining the quantity of PAR; and
wherein the ELISA comprises after a contacting step one or more washing steps.

23. Method according to claim 22, wherein detecting the quantity of second antibody comprises the steps of:
contacting the second antibody, bound to PAR bound to the first antibody, with a third antibody which is an antibody capable of binding the polyclonal antibody and which is not capable of binding the monoclonal antibody, and wherein the third antibody is conjugated to an enzyme
providing a substrate for the conjugated enzyme;
contacting the conjugated enzyme with the substrate and allowing the conversion of the substrate;
measuring the conversion of substrate thereby detecting the quantity of second antibody bound to PAR.

24. Method according to claim 23, wherein the enzyme is horse radish peroxidase.

25. Method according to claim 24, wherein the substrate is a chromogenic substrate or a chemiluminescent substrate.

26. Method according to claim 25, wherein the substrate is selected from the group consisting of TMB (3,3′,5,5′-tetramethylbenzidine), DAB (3,3′-diaminobenzidine),
ABTS (2,2′-azino-bis(3-ethylbenzothiazoline-6-sulphonic acid).

27. A Kit for determining PARP activity, comprising a PARP activating compound and written instructions to perform a method according to claim 1.

28. A Kit for determining PARG activity, comprising a PARP activating compound and written instructions to perform a method according to claim 21.

29. (canceled)
